(19) United States
(12) Patent Application Publication
(10) Pub. No.: US 2006/0121572 A1
(43) Pub. Date: Jun. 8, 2006

(54) Production of heterologous proteins in Caulobacter

(76) Inventors: John Smit, Richmond (CA); John F. Nomellini, Richmond (CA)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/284,238

(22) Filed: Nov. 21, 2005

Related U.S. Application Data
(60) Provisional application No. 60/630,331, filed on Nov. 23, 2004.

(51) Int. Cl. C07K 14/195; C07H 21/04; C12P 21/06; C12N 1/21; C12N 15/74

(52) U.S. Cl. 435/69.1; 435/320.1; 435/252.3; 530/350; 536/23.7

(57) ABSTRACT

The present invention has identified the Caulobacter RsaFa and RsaFb proteins as the OMP components of the S-layer protein secretion machinery. The present invention has also identified that an enhanced production of a heterologous protein from Caulobacter can be achieved by increased production of RsaFa and/or RsaFb. Expression vectors, genetically engineered Caulobacter strains, as well as methods are provided that are useful for producing a heterologous protein in Caulobacter.

### CROSS-REFERENCE TO RELATED APPLICATION

This application claims the benefit of U.S. Provisional Application No. 60/630,331, filed on Nov. 23, 2004.

### FIELD OF THE INVENTION

This invention relates to the use of Caulobacter as a host organism and the Caulobacter surface layer protein (S-layer protein) secretion machinery for the production of heterologous polypeptides.

### BACKGROUND OF THE INVENTION

Crystalline surface protein layers (S-layers) are common in many genera of microorganisms, including Gram-negative bacteria, Gram-positive bacteria and Archaea. S-layers may function as protective barriers and molecular sieves, promote cell adhesion and surface recognition, and maintain cell shape and envelope rigidity (23). The S-layer of the Gram-negative bacterium Caulobacter crescentus has been shown to act as a physical barrier to a Bdellovibrio-like parasitic bacterium (26).

The C. crescentus S-layer is a two-dimensional hexagonal array composed of the 98-kDa protein RsaA (41, 42) that is anchored to the outer membrane via an interaction with smooth lipopolysaccharide (S-LPS) (4, 48). Production of the C. crescentus S-layer has been estimated to be 10-12% of total cell protein with approximately 40,000 RsaA subunits attached to the surface of the cell (5, 8, 41). RsaA synthesis occurs without need for induction and the protein is produced continuously throughout the cell cycle (17, 39).

While most of the characterized S-layer transport systems for Gram-negative bacteria involve type II secretion (in which an N-terminal signal system directs export across the inner membrane using the general secretory pathway (GSP) and secretion from the bacterium then occurs via a protein specific mechanism), the S-layer protein of C. crescentus is secreted by a type I mechanism (3). This mechanism is also found for the S-layer of Campylobacter fetus (46) and the S-layer-like protein (S1aA) in Serratia marcescens (24). The sheer amount of protein secreted by the rsaA secretion apparatus suggests a well organized transport system.

Type I secretion is a sec-independent pathway where protein is transported from the cytoplasm across the inner and outer membranes without interaction with the periplasm. Type I secreted proteins utilize an uncleaved C-terminal secretion signal and, in general, the last 60 amino acids of the protein are sufficient for secretion (6). A type I secretion apparatus is composed of three components. The ATP-binding cassette (ABC) transporter, which likely forms a dimer, resides in the inner membrane, engaging the C-terminal sequence of the substrate protein and hydrolyzing ATP during transport. The membrane fusion protein (MFP) is anchored in the inner membrane by a single transmembrane domain, as well as binding to the ABC transporter, and may span the periplasm (45). The MFP is thought to interact with the ABC transporter protein and a trimer of the outer membrane protein (OMP), to form a channel from the cytoplasm to the outside of the cell (25).

For most type I systems, the ABC transporter and MFP genes are immediately 3′ of the gene for the secreted protein, whereas the OMP gene location can vary. In some cases, the genes for all three transport components are immediately adjacent to the substrate gene(s) (13, 27, 46). In other type I systems, only the ABC-transporter and MFP genes are located next to the substrate gene (28,30) and the OMP gene lies far from the others. TolC, the OMP for the E. coli α-hemolysin (HlyA) system, is an example (50).

The E. coli HlyA system is the best characterized type I secretion system (12, 19, 31). The structure of the OMP (TOlC) has been solved to 2.1 Å (25) and TolC has been shown to be multifunctional, interacting with numerous efflux systems including the HlyA, AcrA, and CvaA systems (18, 22, 50). Thus, it appears to interact with inner membrane translocases involved in drug and cation efflux, as well as protein secretion. It has been suggested that the reason TolC is multifunctional may be that its gene does not belong to any export operon (2).

The RsaA secretion system has the expected characteristics of a type I secretion system. The secretion signal has been localized to the C-terminal 82 amino acids of RsaA (11). The ABC transporter (RsaD) and MFP (RsaE) have been characterized (3) and the rsaD and rsaE genes are found immediately downstream of rsaA. However, an OMP candidate did not immediately follow the rsaE gene, and despite repeated efforts, no Tn5 mutations were found in any OMP-like genes. This suggested either that the OMP was essential or that there were multiple functional OMP genes for this type I system.

As part of a study characterizing the S-LPS genes that were adjacent to rsaD and E (and involved with attachment of the S-layer), a putative OMP gene (rsaFa) was identified (4). Another study (35), however, declared that rsaFa was not an OMP, since disruption of the gene did not abolish RsaA secretion.

In accordance with the present invention, the products of rsaFa and rsaFb have been characterized and confirmed as the only OMP proteins involved in S-layer protein transport. Based on the characterization of RsaFa and RsaFb, the present invention provides vectors, strains and methods useful for expressing a heterologous protein in Caulobacters.

### SUMMARY OF THE INVENTION

The present invention is premised on the identification of the Caulobacter RsaFa and RsaFb proteins as the OMP components of the S-layer protein secretion machinery, and on the recognition that an enhanced production of a heterologous protein from Caulobacter can be achieved by manipulating the S-layer protein secretion machinery, in particular, by increased production of RsaFa and/or RsaFb.

In one aspect of the present invention, an expression vector is provided that contains a nucleotide sequence coding for a Caulobacter RsaFa or RsaFb protein and that is capable of expressing the encoded RsaFa or RsaFb protein in a Caulobacter host.

In a preferred embodiment, the expression vector contains a nucleotide sequence coding for a Caulobacter crescentus RsaFa or RsaFb protein. Examples of Caulobacter crescentus RsaFa and RsaFb proteins are set forth in SEQ ID NO: 2 and SEQ ID NO: 4, respectively, which are encoded by the nucleotide sequence as set forth in SEQ ID NO: 1 and SEQ ID NO: 3, respectively.

In other embodiments, nucleotide sequences that code for a naturally-occurring form of Caulobacter crescentus RsaFa or RsaFb other than SEQ ID NO: 2 and SEQ ID NO: 4, are used in the expression vector of the present invention. In addition, nucleotide sequences coding for a functional derivative or fragment of RsaFa or RsaFb can be used in the expression vector. Moreover, nucleotide sequences that code for a RsaFa or RsaFb protein of a Caulobacter species other than Caulobacter crescentus, or a functional derivative or fragment thereof, can be used in the expression vector as well.

The RsaFa or RsaFb-coding sequence on the expression vector of the present invention is usually placed in an operable linkage to a promoter and a 3′ termination sequence that are functional in the Caulobacter host to achieve expression of the encoded protein. The vector generally also includes a selectable marker gene for convenient selection of transformants.

In one embodiment, the expression vector that contains a RsaFa or RsaFb-coding sequence is a replicative vector that can be maintained in a Caulobacter host in multiple copies.

In another embodiment, the expression vector is an integrative vector that can mediate integration of the RsaFa or RsaFb-coding sequence into the host Caulobacter genome.

In another aspect of the present invention, a genetically engineered Caulobacter strain is provided that produces at least one of the RsaFa or RsaFb protein at an elevated level compared to the strain without genetic engineering.

In a preferred embodiment, the genetically engineered Caulobacter strain produces RsaFa at an elevated level. In another preferred embodiment, the genetically engineered Caulobacter strain produces both RsaFa and RsaFb at elevated levels.

The genetic engineering can be achieved by transforming a Caulobacter strain with an expression vector, either a replicative vector or an integrative vector, that carries a nucleotide sequence coding for a RsaFa or RsaFb protein or a functional derivative or fragment thereof, operably linked to a promoter sequence.

A preferred Caulobacter species for use in the present invention is Caulobacter crescentus, although all species or strains of Caulobacter can be used in the genetic engineering.

In another embodiment, a genetically engineered Caulobacter strain, which produces at least one of the RsaFa or RsaFb protein at an elevated level, is further transformed with an expression vector that carries a nucleotide sequence coding for a heterologous protein. The nucleotide sequence coding for the heterolgous protein is placed in frame to a nucleotide sequence coding for a Caulobacter S-layer protein (RsaA) or a portion thereof that contains a secretion signal of the S-layer protein. The nucleotide sequence coding for the heterologous protein-RsaA fusion is operably linked to a promoter functional in the genetically engineered Caulobacter host. The expression vector that carries the heterologous protein-RsaA fusion coding sequence can be a replicative vector or an integrative vector.

In a further aspect of the present invention, methods for enhanced production of a heterologous polypeptide are provided, which employs the genetically engineered Caulobacter strains of the present invention.

### BRIEF DESCRIPTION OF THE DRAWINGS

FIG. 1A-1C. A. RsaFa and RsaFb levels in wild-type and rsaF knockout strains. Chemiluminescent anti-RsaFa Western blots of whole-culture protein preparation samples are shown. Lanes: 1, NA1000; 2, JS1008; 3, JS1007; 4, JS1009. Arrows indicate RsaFa and RsaFb. B. Effect of disruption of the rsaF genes on S-layer secretion. Chemiluminescent Western blots of low-pH-extracted protein are shown. Lanes: 1, JS1003; 2, JS1009; 3, JS1007; 4, JS1008; 5, NA1000. RsaA is indicated by an arrow. C. Determination of internal levels of RsaA in the rsaF double knockout. Whole-cell protein preparations were visualized by colorimetric Western blotting with polyclonal anti-188/784 RsaA antibody. Lanes: 1, NA1000, 2, JS1009; 3, JS1001; 4, JS1003. An arrow indicates RsaA.

FIG. 2A-2B. A. Complementation of JS1009 with the rsaF genes. Whole-culture preparations were compared by Western blotting and visualized by chemiluminescence using the anti-RsaFa antibodies. Lanes: 1, NA1000; 2, JS1009; 3, JS1009; 3, JS1009 rsaFb; 4, JS1009 rsaFa. B. Complementation of the rsaF genes in trans restores S-layer secretion. Chemiluminescent Western blotting of low-pH-extracted protein using anti-188/784 RsaA antibodies is shown. Lanes: 1, NA1000; 2, JS1009 rsaFa; 3, JS1009 rsaFb; 4, JS1009.

FIG. 3. RsaA production and RsaA secretion levels are comparable. Whole-culture preparations run on SDS-12% PAGE gels were transferred to a nitrocellulose membrane and analyzed with anti-188/784 RsaA antibodies. Lanes: 1, NA1000; 2, JS1008; 3, JS1007; 4, JS1009.

FIG. 4. Impeded RsaA transport in rsaF mutant (JS1009) and RsaA mutant (JS1003 Hps12furin). Whole-cell preparations were loaded on SDS-12% PAGE gels and compared by Western blotting with anti-188/784 RsaA antibodies. The NA1000 strain was subjected to low-pH extraction before whole-cell preparations were made to minimize the levels of S-layer. No RsaA breakdown products are present in the mutants. Lanes: 1, NA1000; 2, JS1009; 3, JS1003::pWB9:723/furin; 4, JS1003.

FIG. 5A-5B. Colloidal gold labeling of surface-displayed RsaF. Surface-displayed RsaF (RsaFa and RsaFb) was determined for the JS1001 strains over expressing rsaFa or rsaFb by electron microscopy with anti-RsaFa and colloidal gold labeling. A. JS1001 shows wild-type levels of RsaF with moderate labeling. B. The JS1001 rsaFa strain shows a significant increase in surface display of RsaF. Note that only about 20% of the JS1001 rsaFa cells showed a significant increase in RsaF display.

FIG. 6. Effect of RsaF overexpression in the JS1001 strain. Whole-culture preparations were run on SDS-12% PAGE gels and compared by Western blotting with anti-188/784 antibodies, Lanes: 1, JS1001; 2, JS1001 rsaA; 3, JS1001 rsaFa; 4, JS1001 rsaFa rsaA.

### DETAILED DESCRIPTION OF THE INVENTION

The present inventors have identified RsaFa and RsaFb as the outer membrane protein (OMP) components of the RsaA secretion machinery in Caulobacter. Specifically, the present inventors have determined that elimination of either RsaFa or RsaFb results in decreased RsaA secretion, and that elimination of both RsaFa and RsaFb results in a complete loss of RsaA secretion. Further, the present inventors have determined that overexpression of one of RsaFa and RsaFb, absent the expression of the other, does not restore the secretion of RsaA to wild-type levels. However, if both RsaFa and RsaFb are present in the cells, overexpression of one leads to levels of RsaA secretion significantly above wild-type levels. The present inventors have also discovered that enhanced expression of a heterologous protein in a Caulobacter host can be achieved by placing the heterologous protein in an operable linkage to a secretion signal of RsaA, in conjunction with overexpressing at least one of RsaFa and RsaFb in the Caulobacter host.

As used herein, the designations “RsaA”, “RsaFa” and “RsaFb” represent the respective proteins, whereas the designations “rsaA”, “rsaFa” and “rsaFb” (i.e., in italics and with the first letter in lower case) represent the respective nucleic acid molecules.

In one aspect of the present invention, an expression vector is provided that contains a nucleotide sequence coding for a Caulobacter RsaFa or RsaFb protein and that is capable of expressing the encoded RsaFa or RsaFb protein in a Caulobacter host.

In accordance with the present invention, preferred nucleotide sequences to be employed in the expression vector include nucleotide sequences coding for a Caulobacter crescentus RsaFa or RsaFb protein. An example of a Caulobacter crescentus RsaFa protein is set forth in SEQ ID NO: 2. An example of a Caulobacter crescentus RsaFb protein is set forth in SEQ ID NO: 4. Accordingly, preferred nucleotide sequences to be employed in the expression vector include nucleotide sequences coding for a protein composed of SEQ ID NO: 2 or SEQ ID NO: 4. For example, such nucleotide sequences are set forth in SEQ ID NO: 1 and SEQ ID NO: 3.

Further, nucleotide sequences that code for naturally-occurring forms of the Caulobacter crescentus RsaFa and RsaFb proteins other than SEQ ID NO: 2 and SEQ ID NO: 4, can also be used in the expression vector of the present invention.

In addition, nucleotide sequences that code for a functional derivative or fragment of RsaFa and RsaFb as set forth in SEQ ID NO: 2 and SEQ ID NO: 4, respectively, can also be used. By “a functional derivative or fragment” is meant a derivative or a part of the RsaFa or RsaFb protein that substantially retains the functional activity of the native, full-length protein in the secretion process of the S-layer protein. By “substantially” is meant at least about 50%, or preferably, at least 75% or more of the OMP activity of the full-length RsaFa or RsaFb protein.

Additionally, nucleotide sequences to be employed in the expression vector of the present invention can be a nucleotide sequence coding for a RsaFa or RsaFb protein of a Caulobacter species other than Caulobacter crescentus, which species naturally produces S-layer proteins. Caulobacter species that produce S-layer proteins can be readily identified and isolated by those skilled in the art. Examples of non-crescentus Caulobacter species that produce S-layer proteins and are suitable to provide rsaFa and rsaFb sequences include those described by MacRae and Smit (Applied Environemental Microbiol. 57: 751-758, 1991), Walker et al. (J. Bacteriol. 174: 1783-1792, 1992), WO 00/49163, and Luga et al. (Can. J. Microbiol. 50: 1-16, 2004), all of which are incorporated herein by reference; for example, FWC1, FWC8, FWC9, FWC17, FWC19, FWC28, FWC32, FWC39 and FWC42, as described in WO 00/49163. In particular, the present invention contemplates the use of nucleotide sequences that code for a protein of at least 80%, preferably, 90%, or more preferably, 95%, identical with SEQ ID NO: 2 or SEQ ID NO: 4. In addition, non-C. crescentus nucleotide sequences can be used that hybridize under stringent conditions to the complement of SEQ ID NO: 1 or SEQ ID NO: 3. Similarly, nucleotide sequences that encode functional derivatives and fragments of a non-C. crescentus RsaFa or RsaFb protein can also be used.

By “stringent conditions” are meant to include conditions of low, medium and high stringencies. Preferably, the conditions are high stringency conditions which include, for example, prehybridization at a temperature of about 60-65° C. in buffer containing about 6×SSC, 50 mM Tris-HCl (pH 7.5), 1 mM EDTA and 500 μg/ml denatured salmon sperm DNA; hybridization at about 60-65° C. in prehybridization mixture containing 100 μg/ml denatured salmon sperm DNA; and washing at about 37° C. in a solution containing 2× SSC, 0.01% PVP, 0.01% Ficoll, and 0.01% BSA, followed by a wash in 0.1× SSC at about 50° C.

The RsaFa or RsaFb-coding sequence on the expression vector of the present invention can be placed in an operable linkage to a promoter and a 3′ termination sequence. The promoter can be the native rsaFa or rsaFb promoter, or any other promoter that is functional in the Caulobacter host to achieve expression of the encoded protein. Preferably, the vector also includes a selectable marker gene for convenient selection of transformants.

According to the present invention, the expression vector that contains a RsaFa or RsaFb-coding sequence can be either a replicative vector or an integrative vector.

A replicative vector refers to a vector that can be maintained in host cells through replication independent of the host genome. In a preferred embodiment, the expression vector of the present invention is a replicating plasmid that can be maintained in a Caulobacter host in multiple copies to provide enhanced expression of the encoded RsaFa or RsaFb protein. Multi-copy plasmid vectors include those high copy number plasmids that are present at about 20-25 copies per cell or more, and those moderate copy number plasmids that are present at about 4-8 copies per cell. Multi-copy plasmid vectors are available from various commercial sources, e.g., Invitrogen, and are also described in the examples hereinbelow.

An integrative vector refers to a vector that mediates the integration of a sequence in the vector into the chromosomes of the host cells. For the purpose of the present invention, an integrative vector generally includes a RsaFa or RsaFb-coding sequence, that is desired to be integrated into a host chromosome, preferably linked to a promoter sequence. Such a promoter-rsaF sequence is sandwiched between nucleotide sequences that correspond to 5′ or 3′ portions of a target genomic DNA, preferably 5′ or 3′ portions of a non-essential region, on a chromosome of the host Caulobacter. An example of a useful non-essential region of the Caulobacter genome is the xylose utilization operon. The genes in this region enable Caulobacter to grow with xylose as a sole carbon source. The xylX gene is the first gene in this operon, and disruption by insertion of additional DNA is simply accompanied by the lack of the ability to grow on minimal media with xylose as the sole carbon source. An integrative vector, which contains a promoter-rsaF sequence flanked by nucleotide sequences that correspond to 5′ or 3′ portions of a target genomic DNA, can be introduced into the host cells by transformation, integrated into the target genomic DNA chromosome site via homologous recombination, resulting in the insertion of the promoter-rsaF sequence into the host chromosome. To facilitate selection of integration events, an integrative vector typically also includes a selectable marker gene that confers a selectable phenotype (e.g., antibiotic resistance) on the transformed host cells. More than one selectable marker genes can be placed on an integrative vector, and depending upon the position of a marker on the integrative vector relative to 5′ and 3′ target sequences, the marker can serve as a positive or a negative selection marker of integration events. Preferred integrative vectors of the present invention also include those that lack the ability to replicate in host cells, thereby favoring the selection of transformants with successful integration.

In another aspect of the present invention, a genetically engineered Caulobacter strain is provided that produces at least one of the RsaFa or RsaFb protein at an elevated level.

By “elevated” or “enhanced” in reference to the production of RsaFa and RsaFb is meant that as a result of genetic engineering in accordance with the present invention, a Caulobacter strain produces at least 10%, preferably 15%, more preferably at least 20%, or even more preferably at least 25% or 40%, more RsaFa or RsaFb proteins as compared to the strain without the genetic engineering.

In a preferred embodiment, the present invention provides a genetically engineered Caulobacter strain that produces RsaFa at an elevated level.

In another preferred embodiment, the present invention provides a genetically engineered Caulobacter strain that produces RsaFa and RsaFb at elevated levels.

According to the present invention, a Caulobacter strain can be genetically engineered as follows to achieve enhanced production of RsaFa or RsaFb. In one embodiment, a Caulobacter strain can be transformed with a replicative expression vector, as described above, that contains a nucleotide sequence that codes for a RsaFa or RsaFb protein or a functional derivative or fragment thereof. The RsaFa or RsaFb-coding sequence is operably linked to a promoter sequence that directs the expression of the encoded protein in the host Caulobacter strain. The replicative vector typically carries a selectable marker gene and is preferably present in the host at multiple copies in order to achieve desirable, elevated production of RsaFa or RsaFb. In an alternative embodiment, a Caulobacter strain can be transformed with an integrative expression vector, as described above. The integrative vector contains a nucleotide sequence that codes for a RsaFa or RsaFb protein or a functional derivative or fragment thereof, which is linked to a promoter sequence. Transformants are then selected for those in which the promoter-rsaF DNA segment has integrated into the chromosome of the host Caulobacter.

Caulobacter suitable for use as a host of the genetic engineering in accordance with the present invention includes include all species or strains of Caulobacter that naturally produce S-layer proteins. A preferred Caulobacter species for use is Caulobacter crescentus. In other embodiments, Caulobacter species other than Caulobacter crescentus are used as a host. Examples of non-crescentus Caulobacter species that naturally produce S-layer proteins include those described by MacRae and Smit (Applied Environemental Microbiol. 57: 751-758, 1991), Walker et al. (J. Bacteriol. 174: 1783-1792, 1992), WO 00/49163, and Luga et al. (Can. J Microbiol. 50: 1-16, 2004), all of which are incorporated herein by reference; for example, FWC1, FWC8, FWC9, FWC17, FWC19, FWC28, FWC32, FWC39 and FWC42, as described in WO 00/49163.

Both Caulobacter strains that form an S-layer and Caulobacter strains that are incapable of forming an S-layer can be used as a host. In some cases, it may be desirable to use strains that are incapable of forming an S-layer, such as those that shed the S-layer protein upon secretion. Examples are the S-layer negative C. crescentus mutants CB2A and CB15AKSac, described in Smit et al. (J. Bacteriol. 160: 1137-1145, 1984) and Edwards et al. (J. Bacteriol. 173:5568-5572, 1991). Examples of shedding strains are CB15Ca5 and CB15Ca10, described in Edwards et al. (1991), and the smooth lipopolysaccharide deficient mutants described in Walker et al. (J. Bacteriol. 176: 6312-6323, 1994).

The genetically engineered Caulobacter strains of the present invention, which produce RsaFa or RsaFb at elevated levels, are especially useful for enhanced production of a heterologous protein.

By “elevated” or “enhanced” in reference to the production of a heterologous protein is meant that as a result of genetic engineering (i.e., enhanced production of RsaFa or RsaFb) in accordance with the present invention, a Caulobacter strain, once transformed with an expression vector coding for the heterologous protein, can produce at least 10%, preferably 15%, more preferably at least 20%, or even more preferably at least 25% or 40%, more of the heterologous protein as compared to the strain prior to the genetic engineering relating to enhanced production of RsaFa or RsaFb.

A “heterologous protein” as used herein refers to a protein that is not present in, i.e., foreign to, the host Caulobacter strain, in which the protein is to be produced. Heterologous proteins that can be produced at elevated levels by the genetically engineered Caulobacter strains are not limited to any particular proteins, and include bacterial, fungal, viral or mammalian proteins or antigens. Examples of heterologous proteins suitable for production in the genetically engineered Caulobacter strains are Bacillus amyloliquefaciens α-amylase, S. cerevisiae invertase, Trypanosoma cruzi trans-sialidase, HIV envelope protein, influenza virus A haemagglutinin, influenza neuramimidase, Bovine herpes virus type-1 glycoprotein D; proteins, a protein of a mammalian origin, such as human proteins, growth factors or receptors, e.g., human angiostatin, human B7-1, B7-2 and B-7 receptor CTLA-4, human tissue factor, growth factors (e.g., platelet-derived growth factor), tissue plasminogen activator, plasminogen activator inhibitor-I, urokinase, human lysosomal proteins such as α-galactosidase, plasminogen, thrombin, factor XIII; immunoglobulins or fragments (e.g., Fab, Fab′, F(ab′)₂) of immunoglobulins, Cellumonoasfimi exocellulase, Infectious Hematopoietic necrosis virus glycoprotein segments, Infectious Pancreatic Necrosis Virus glycoprotein segments, immunoglobulin binding domains of the Protein G of Peptostreptococcus, portions of Escherichia coli beta-galactosidase, Escherichia coli alkaline protease, Bacillus anthracis Anthrax Protective Antigen, and Intimin binding domains of Tir proteins from enteropathogenic Escherichia coli.

To achieve elevated production of a heterologous protein in a Caulobacter strain, a nucleotide sequence coding for the heterolgous protein is placed in frame to a nucleotide sequence coding for a Caulobacter S-layer protein (RsaA) or a portion thereof that contains a secretion signal of the S-layer protein.

The rsaA sequence can be of an origin of the same or different Caulobacter species in which the desired heterologous protein will be expressed. Preferably, the S-layer protein is from the same Caulobacter species as the rsaFa or rsaFb sequence that is introduced into the host for enhanced expression. For example, in a preferred embodiment, a rsaA sequence from C. crescentus is employed in conjunction with an expression vector carrying a C. crescentus rsaFa or rsaFb sequence. A preferred rsaA sequence from C. crescentus is disclosed in U.S. Pat. No. 5,500,353. In other embodiments, an rsaA sequence is from a Caulobacter species other than Caulobacter crescentus, for example, those non-crescentus Caulobacter species or strains described by MacRae and Smit (Applied Environemental Microbiol. 57: 751-758, 1991), Walker et al. (J. Bacteriol. 174: 1783-1792, 1992), WO 00/49163, and Luga et al. (Can. J. Microbiol. 50: 1-16, 2004), all of which are incorporated herein by reference; for example, FWC1, FWC8, FWC9, FWC17, FWC19, FWC28, FWC32, FWC39 and FWC42, as described in WO 00/49163.

According to the present invention, the secretion signal localized within the C-terminal portion of an S-layer protein interacts with the RsaFa and RsaFb proteins to direct secretion of a heterologous protein, and is sufficient to support an elevated secretion of the heterologous protein in a host with elevated production of one or both of RsaFa and RsaFb.

In a preferred embodiment, the nucleotide sequence coding for a heterolgous protein is placed in frame, typically 5′, to a nucleotide sequence coding for a protein that includes at least the C-terminal 82 amino acids of a Caulobacter S-layer protein, preferably the S-layer protein of C. crescentus. In a specific embodiment, the nucleotide sequence coding for a heterolgous protein is placed in frame and 5′ to a nucleotide sequence coding for the C-terminal 336 amino acids of a C. crescentus S-layer protein.

The nucleotide sequence coding for the heterologous protein-RsaA fusion is also operably linked to a promoter that is functional in Caulobacter host, and is placed on an expression vector for convenient transformation into a Caulobacter host. Similar to the expression vector that carries a RsaF coding sequence, the expression vector that carries a heterologous protein coding sequence can be a replicative vector (e.g., a high or moderate copy number plasmid) or an integrative vector.

To achieve enhanced production of a heterologous protein, the expression vector carrying the nucleotide sequence coding for the heterologous protein can be introduced into a Caulobacter host prior to, simultaneous with, or subsequent to the introduction of the expression vector carrying a RsaF coding sequence. In fact, the nucleotide sequence coding for a heterologous protein can be included in the same expression vector as the RsaF coding sequence for one-step transformation into Caulobacter.

Accordingly, genetically engineered Caulobacter strains are provided by the present invention, which produces elevated levels of one or both RsaFa or RsaFb, and which also produces a heterologous protein of interest.

In a further aspect of the present invention, methods for producing a heterologous protein by employing the vectors and/or Caulobacter strains as described hereinabove are provided. The heterologous protein produced from the genetically engineered Caulobacter strain can be conveniently purified as described in U.S. Pat. No. 5,500,353, U.S. Pat. No. 6,210,948 and International Application PCT/CA99/00637.

The present invention is further illustrated by and by no means limited to the following examples.

### EXAMPLE 1

### Materials and Methods

Strains, Plasmids, and Growth Conditions

Strains and plasmids used in the following examples are listed in Table 1. E. coli DH5α was grown at 37° C. in Luria broth (1% tryptone, 0.5% NaCl, 0.5% yeast extract) with 1.3% agar for plates. C. crescentus strains were grown at 30° C. in PYE medium (0.2% peptone, 0.1% yeast extract, 0.0 1% CaCl₂, 0.02% MgSO₄) with 1.2% agar for plates. Ampicillin (Ap) was used at 50 μg/ml, kanamycin (Km) was used at 50 μg/ml and 25 μg/ml, chloramphenicol (Cm) at 20 μg/ml and 2 μg/ml, and streptomycin (Sm) at 50 μg/ml and 10 μg/ml in E. coli and C. crescentus, respectively.

TABLE 1
Bacterial strains and plasmids

| Strain or plasmid | Relevant characteristics^{a} | Reference or source |
|---|---|---|
| Bacterial strains | | |
| C. crescentus | | |
| NA1000 | Ap^{r} syn-1000; variant of wild-type strain CB15 that synchronizes well | ATCC 19089 |
| JS1001 | S-LPS mutant of NA1000, sheds S-layer into medium | Edwards and Smit (14) |
| JS1003 | NA1000 wit rsaA interrupted by KSAC Km^{r} cassette | Edwards and Smit (14) |
| JS1007 | Sm^{r}; NA1000 rsaFa strain | Example 1 |
| JS1008 | Cm^{r}; NA1000 rsaFb strain | Example 1 |
| JS1009 | Cm^{r} Sm^{r}; NA1000 rsaFa rsaFb strain | Example 1 |
| E. coli DH5α | recA endA | Invitrogen |
| Plasmids | | |
| pHP45Ω | Ap^{r} Sm^{r}; source of SM cassette removed as-SmaI fragment | Fellay et al. (15) |
| pBSKIIEEH | Ap^{r}; modified pBSKSII cloning vector with EcoRI-, EcoRV- | Example 1 |
| | HindIII-modified MCS | |
| pBSKHESH | Ap^{r}; modified pBSKSII cloning with Eco-RI-, StuI-, HindIII- | Example 1 |
| | modified MCS | |
| pTZ18UCHE | Cm^{r}; cloning vector | Example 1 |
| pTZ18UCHE:rsaFbΔNΔC | Cm^{r}; cloning vector with rsaFb fragment missing N and C termini | Example 1 |
| pBSKIIEEH:rsaFaΩSm | Ap^{r} Sm^{r}; rsaFa gene fragment with SM cassette inserted at PstI site | Example 1 |
| pBBR4 | Km^{r}; broad-host-range plasmid derived from pBBR1 | Example 1 |
| pBBR4:rsaFa | RsaFa⁺ Km^{r}; rsaFa gene inserted EcoRI/BamHI in pBBR4 | Example 1 |
| pBBR4:rsaFb | rsaF⁺ Km^{r}; rsaFb gene inserted EcoRI/BamHI in pBBR4 | Example 1 |
| pWB9:rsaAΔP | Cm^{r} Sm^{r}; rsaA gene and rsaA promoter strain | Bingle et al. (9) |
| pWB9Hps12 | Cm^{r} Sm^{r}; rsaA containing BamHI site at aa 723 | Bingle et al. (10) |
| pWB9:723/furin | Cm^{r} Sm^{r}; rsaA containing furin cleavage site (RKKR) in BamHI site | Example 1 |
| pGEX4T3 | Ap^{r}; GST-tagged expression vector | Amersham |
| pGEX4T3:rsaFa | Ap^{r}; GST-tagged expression vector with in-frame BamHI-EcoRI rsaFa gene | Example 1 |
| pK18mobsacB | Km^{r} Suc^{s}; E. coli-based suicide vector | Schafer et al. (37) |
| pK18mobsacB:rsaFaΩSm | Km^{r} Suc^{s}; E. coli-based suicide vector with rsaFaSm fragment | Example 1 |

^{a}MCS, multiple cloning site;
Suc^{s}, sucrose sensitivity;
KSAC, Km^{r} cassette derived from transposon Tn908.

Plasmid and DNA Manipulations

Standard methods of DNA isolation and manipulation were used (36). Electroporation of C. crescentus was performed as previously described (20). All PCR products were generated using Platinum Pfx DNA polymerase (Invitrogen) following the manufacturer's suggested protocols. NA1000 chromosomal DNA was used as the template for all PCR products. Chromosomal DNA was isolated with standard phenol/chloroform extraction methods (36).

All primers used in this study are listed in Table 2. The PBSKIIEEH vector was constructed from the pBSKII (Stratagene). The BssHI fragment containing the multiple cloning site was removed and replaced with annealed oligonucleotides EEH-1 and EEH-2 forming EcoRI, EcoRV and HindIII sites. pBSKIIESH was created similarly to pBSKIIEEH with annealed oiigonucleotides ESH-1 and ESH-2 forming EcoRI, StuI and HindIII sites.

TABLE 2
Primers

| | | |
|---|---|---|
| | SEQ | |
| | ID | |
| Primer | NO | Sequence |

| | | |
|---|---|---|
| EEH-1 | 5 | 5′-CGCGCTGAATTCGGATATCTTAAGCTTGG-3′ |
| EEH-2 | 6 | 5′-CGCGCCAAGCTTAAGATATCCGAATTCAG-3′ |
| ESH-1 | 7 | 5′-CGCGCTGAATTCGAGGCCTTTAAGCTTGG-3′ |
| ESH-2 | 8 | 5′-CGCGCCAAGCTTAAAGGGCTCGAATTCAG-3′ |
| rsaFa-1 | 9 | 5′-CGCGGATCCATGCGAGTGCTGTCGAAAGTTCTGT |
| | | C-3′ |
| rsaFa-2 | 10 | 5′-CCGGGAATTCTAGTTGCGGGGCGCGGTCTGGA |
| | | C-3′ |
| rsaFb-1 | 11 | 5′-CGCGGATCCATGTTGATGTCGAACCGTCGACGG |
| | | G-3′ |
| rsaFb-2 | 12 | 5′-CCGGGAATTCTATTTCGAGCCGCTCGGGGGCT |
| | | T-3′ |
| rsaFbNC-1 | 13 | 5′-GAAGCCGACGTGCTGTCT-3′ |
| rsaFbNC-2 | 14 | 5′TGTAGGAGGTTTTCGGGTCA-3′ |
| 1060 | 15 | 5′-GAGGCCTAGTACTCTGTCAGACCAAGTTTACTCA |
| | | TA3′ |
| 1920 | 16 | 5′-GAGGCCTACTCTTCCTTTTTCAATATTATTGA |
| | | A-3′ |
| CHE-1 | 17 | 5′GGAAGATCTGTTAACTTTTCAGGAGCTAAGGAAGC |
| | | T3′ |
| CHE-2 | 18 | 5′-GAAGATCTGTTAACACAATAACTGCCTTAAAAAA |
| | | ATTA3′ |
| RKKR-1 | 19 | 5′-TCGAGACCCGATGCGCAAGAAACGGG-3′ |
| RKKR-2 | 20 | 5′-CCCGTTTCTTGCGCATCGGGTC-3′ |
| rsaFb-I90 | 21 | 5′-GGACGACGCTGACCAGCACCCCCTGCT-3′ |

A PCR product containing the rsaFa gene was generated using primers rsaFa-1 and rsaFa-2. A PCR product containing the rsaFb gene was created using primers rsaFb-1 and rsaFb-2. The PCR products were ligated into the EcoRV site of the pBSKIIEEH vector, forming pBSKIIEEH: rsaFa and pBSKIIEEH: rsaFb, respectively.

pBBR4 was constructed from plasmids pBBR1MCS and pUC4 KISS. The Km fragment from pUC4 KISS was removed using PstI and blunted using T4 polymerase. A 0.3-kbp portion of the Cm^{r}-encoding gene was removed from pBBR1MCS with DraI and replaced with the blunted Km fragment, producing a Km^{r} broad-host-range vector that replicated in C. crescentus. pBBR3 was constructed in a similar manner to pBBR4 using a blunted HindIII cut Sm fragment of pHP45 Ω.

Plasmids pBBR4 rsaFa and pBBR4 rsaFb and pGEX4T3: rsaFa were made using the BamHI-EcoRI fragment from pBSKIIEEH: rsaFa and pBSKIIEEH: rsaFb, respectively.

pBSKIIEEH: rsaFaΩSm was created using the plasmids pBSKIIEEH: rsaFa and pHP45Ω. The Ω-Sm cassette was removed as a SmaI fragment and blunt-end ligated into pBSKIIEEH: rsaFa at a blunted PstI site inside rsaFa. The resulting pBSKIIEEH: rsaFaΩSm was then used to make pK18mobsacB: rsaFaΩSm. The EcoRI-HindIII fragment containing the rsaFaΩSm fragment was cloned into EcoRI-HindIII cut pK18mobsacB plasmid.

A PCR product containing a truncated form of rsaFb missing the N- and C-termini was generated using the primers rsaFbNC-1 and rsaFbNC-2. This PCR product was blunt-end ligated into the StuI site of the pBSKIIESH creating pBSKIIESH: rsaFbΔNΔC. pTZ18UCHE was constructed using inverse PCR with the primers 1060 and 1920 to create the backbone of pTZ18U without the Ap^{r} cassette. The CHE (containing a chloramphenicol-resistance gene) fragment was created as a PCR product using pMMB206 (32) and the primers CHE-1 and CHE-2. The pTZ18U backbone product was cut with StuI and blunt-ligated with HpaI cut CHE fragment creating pTZ18UCHE. The EcoRI-HindIII fragment from pTZ18UCHE was removed and replaced with the EcoRI-HindIII rsaFbΔNΔC fragment from pBSKIIESH: rsaFbΔNΔC.

pWB9: rsaAΔP was described previously (10). The pWB9:723/furin construct was made using the BamHI site at amino acid position 723 (Hps12). Two oligonucleotides, RKKR-1 and RKKR-2, were annealed and ligated into pUC9CXS as an XhoI-StuI fragment in a similar manner as the Type IV pilin epitope (9). The RKKR-containing fragment was released using BamHI and then inserted into the BamHI site at amino acid 723 in rsaAΔP and forward orientation of the fragment was confirmed by selection for Cm resistance. The Cm^{r} cassette was excised using BglII and then ligation of the two complimentary ends. The rsaAΔP723/furin fragment was then removed as an EcoRI-SstI fragment and ligated into EcoRI-SstI cut pWB9KSAC.

Knockout Construction

Knockouts of the two rsaF genes were constructed in the wild-type (S-layer positive) C. crescentusNA1000. The rsaFa gene was disrupted by homologous recombination of an rsaFa gene fragment containing an internal ΩSm cassette using pK18mobsacB: rsaFaΩSm as a suicide vector. PYE Sm/Km plates were used to identify single crossover events and five rounds of sub-culturing were used to encourage a second recombination event. Bacteria were grown on 5% sucrose PYE plates to select the second recombination event. Subsequent replicate plating on PYE Sm and PYE Km plates confirmed the removal of the Km^{r} gene. Colonies were then screened by PCR using the primers rsaFa-1 and rsaFa-2 to determine if the recombination event resulted in restoration of wild-type rsaFa or incorporation of the rsaFaΩSm gene fragment.

The rsaFb gene was disrupted by gene inactivation using an N and C-terminally deleted form of rsaFb. Selection on PYE Cm was used to identify integration of the plasmid into the genome. Colonies were screened by PCR using the primers 1060 and rsaFb-190.

The double rsaF knockout was created by homologous recombination using pK18mobsacB: rsaFaΩSm and JS1008 (rsaFb) competent cells. Screening for homologous recombination of the pK18mobsacB: rsaFaΩSm was done in the same manner as the single rsaFaΩSm knockout except that Cm was used in all media to maintain the rsaFb knockout. PCR confirmation of both rsaF knockouts was performed using the primers and conditions as stated above.

Antibody Production

Antibodies to detect RsaA were raised against a form of RsaA containing only the N- and C-terminal portions of the protein, referred to as 188/784. This internal deletion form of the RsaA protein was previously described in linker mutagenesis studies (10). Essentially, the DNA fragment coding for the N-terminal 1-188 aa of RsaA was removed as an EcoRI-BamHI fragment and the portion coding for the C-terminal 784-1025 aa was removed as a BamnHI-HindIII fragment. The two fragments were ligated together at the BarnHI site and then ligated into EcoRI-HindIII cut pUC8; this maintained the translational frame of the protein. The HindIII cut pUCS: 188/784 was ligated to HindIII cut pKT215 vector and transformed into C. crescentus forming aggregated protein which was used to make antibodies against both termini. Aggregated 188/784 protein was collected and washed with dH₂O to remove residual C. crescentus cells. Aggregates were solubilized with 4 M urea and dialyzed against dH₂O to remove urea. Samples were then injected into a New Zealand white rabbit and serum was collected and processed using standard protocols (34).

Polyclonal antibodies were produced against RsaFa using a GST-tagged protein. The pGEX4T3: rsaFa was expressed in E. coli (DH5α), but only produced inclusion bodies. Inclusion-body isolation was performed by growing cells to 1.0 OD₆₀₀ then pelleted and resuspended in 1×PBS buffer with lysozyme (100 μg/ml) for 1 h at 25° C. then RNaseA (50 μg/ml) and DNase I (1 μg/ml) were added and incubated for an additional hour at 25° C. After incubation, 10% SDS (starting conc.) was added at a 1:1 ratio with SDS-PAGE sample buffer and boiled for 5 min, put on ice for 15 min and centrifuged at 16,000×g for 10 min. The inclusion body pellet was then recovered and solublized using 4 M urea. Protein was dialyzed with several changes of dH₂O for two days to remove the urea. A rabbit was immunized as described above.

Protein Analysis Methods

Surface protein from C. crescentus cells was extracted by low pH extraction as previously described (49). To compare the amounts of surface layer protein extracted from different mutants, cells were normalized and loaded onto sodium dodecyl sulfate-polyacrylamide gels (SDS-PAGE).

Whole-cell-protein preparations were made using normalized cells, cultures were pelleted by centrifugation and pellets were washed twice with 10 mM Tris-HCl pH 8. Cells were resuspended in 10 mM Tris-HCl pH 8 with lysozme at 25° C. for 15 min, then RNase A and DNase I were added (as above) and incubated at 37° C. for 30 min. Equal amounts of whole-cell-protein preparations were loaded onto protein gels. Whole-cell preparation methods were altered for certain strains when required to determine internal levels of S-layer. The wild-type S-layer-positive NA1000 had RsaA attached to its surface and required removal by low pH extraction and subsequent washes of the cell pellet before whole-cell preparations were performed to allow assessment of internal RsaA. To remove secreted RsaA from the S-layer shedding JS1000 strain, culture medium was poured through a fine nylon mesh (350 μm pore size) to remove aggregates so that they did not affect RsaA levels detected in the whole-cell preparation.

Whole-culture preparations were made using normalized cells as above. Whole-culture preparations were not centrifuged, ensuring that any aggregated RsaA was small and well dispersed and included. Cultures were incubated with lysozme, RNase A and DNase I as above. Urea was added to the samples to give a final concentration of 2 M to solubilize any micro-aggregates produced by S-layer shedding strains. Equal amounts of whole-culture-protein preparations were loaded onto SDS-PAGE.

SDS-PAGE and Western Immunoblotting

SDS-PAGE was performed using 7.5% or 12% (as indicated) separating gels. Coomassie-staining of gels and Western immunoblotting were performed using standard protocols (36) and 0.2 μm BioTrace NT nitrocellulose membranes (Pall Biosciences). Western blots were visualized by calorimetric or electro-chemiluminescence (ECL) developing methods. Colorimetric detection was performed as previously described (40). Chemiluminescent blotting was done using the Amersham Biosciences ECL western blotting kit in accordance with the manufacturer's protocol. To generate quantitative values and standard deviations (Xση-1) all data reported for ECL Western blots represent the result of at least three and as many as seven replicates.

Anti-188/784 antibodies were incubated at 1/15,000 for calorimetric and 1/30,000 for chemiluminescent Western blotting. Incubation using primary anti-RsaFa was done at a 1/1,000 dilution for colorimetric and 1/5,000 dilution for chemiluminescent blotting. Kodak X-OMAT LS film was used for visualization of chemiluminescent blots, while spot densitometry was done using a Bio-Rad VersaDoc 5000 system and the Quantity One (V4.3.0) program. Within the exposure limits defined by the software, quantitative densitometry using the VersaDoc 5000 allowed for a linear response over several orders of magnitude.

Bioinformatic Analysis

Sequences were obtained from The Institute for Genomic Research (TIGR) C. crescentus genome database. Protein sequence alignments were made using the BLASTP sequence alignment tools (1) available at the Biology Workbench website. ClustalW alignments of the RsaFa (CC1015), RsaFb (CC1318) and TolC proteins were done using MacVector 6.0.

Electron Microscopy

Protein A-colloidal gold immunolabeling of C. crescentus using the anti-RsaFa antibody and 5 nm protein A-colloidal gold label prepared was performed as previously described (35). The antibody was preabsorbed with JS1009 cells. Cells (1 ml of cells with OD₆₀₀ of 1) were centrifuged at 16000×g for 5 min. The cell pellet was resuspended in 10 mM Tris-HCl pH 8 and disrupted by sonication for 5 sec using a microprobe at maximum power. Sonicated cells were centrifuged at 16000×g for 2 min and the supernatant was discarded. Anti-RsaEa rabbit serum was added to suspend the pellet; the suspension was incubated on ice for 1 h followed by centrifugation at 16000×g for 3 min. The supernatant was used for labeling. Cells were imaged unstained by whole mount transmission electron microscopy.

### EXAMPLE 2

### Identification of Two OMP Genes

Sequences were obtained from The Institute for Genomic Research (TIGR) C crescentus genome database. Protein sequence alignments were achieved using the BLASTP sequence alignment tools (1). Two possible candidates for the OMP proteins with similarity to the E. coli TolC protein sequence were identified in the C. crescentus genome (33). These candidate genes were named rsaFa and rsaFb, the coding sequences of which are set forth in SEQ ID NOS: 1 and 3, respectively. The RsaFa protein (SEQ ID NO: 2) was found to have 23% identity with and 45% similarity to TolC; and the RsaFb protein (SEQ ID NO: 4) was found to have 25% identity with and 47% similarity to the E. coli TolC, as determined by local sequence alignment using using MacVector 6.0. The rsaFa gene (1581 bp) was determined to be located downstream of the rsaADE genes after a gap of 5025 bp coding for five S-LPS-related genes. The rsaFb gene (1452 bp) was determined to be located 322 kb (303 genes) away from rsaFa and flanked by genes of unknown function. The two rsaF genes were determined to have significant similarity with each other, sharing 39% identity and 60% similarity, and could be the result of gene duplication. A BLAST search of the C crescentus genome for other candidate OMPs using RsaFa as a query sequence identified several predicted proteins with moderate e-values, which are believed to be outer membrane lipoproteins.

### EXAMPLE 3

### Disruption of the rsaFa and rsaFb Genes

Knockouts of rsaF genes were constructed by homologous recombination of inactivated rsaF genes in the wild-type C. crescentus strain NA1000. Disruption of rsaFa was performed by gene replacement with an rsaFa gene construct containing an antibiotic-resistance cassette. A mutant resulting from a double cross-over event was confirmed by PCR and designated JS1007. The rsaFb gene was disrupted via insertional inactivation, using an N- and C-terminally deleted rsaFb fragment (rsaFbΔNΔC). The rsaFbΔNΔC fragment was inserted into the chromosome via homologous recombination using a non-replicating plasmid, resulting in two tandem, non-functional copies of rsaFb. Confirmation by PCR showed that there were only disrupted forms of rsaFb in the chromosome. One confirmed mutant was selected and was designated as JS1008. To create the double-knockout strain, rsaFa was disrupted and confirmed in JS1008 in the same manner as for JS1007 and was designated JS1009.

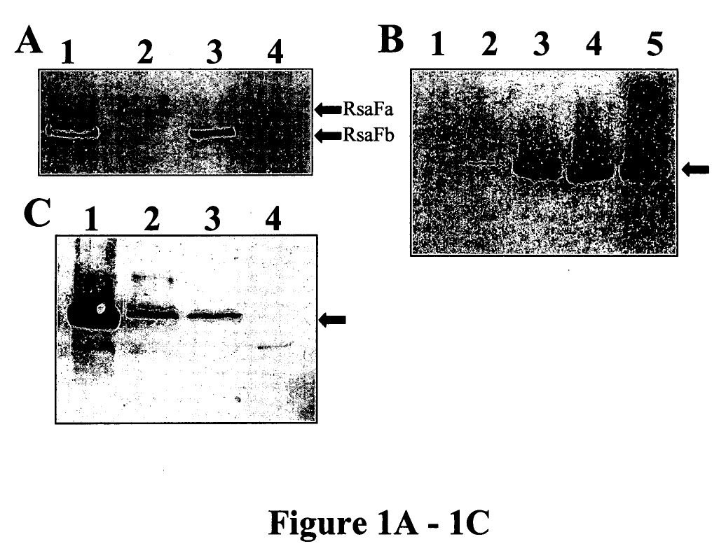

To confirm that RsaFa and RsaFb were not produced in the knockout strains, Western blot analysis was performed using polyclonal anti-RsaFa antibodies. Rabbit polyclonal antibodies were generated against a GST-tagged rsaFa gene product. Cross reactivity between the two RsaF proteins was evident and thus anti-RsaFb antibodies were not raised since the proteins can be differentiated by size (57.5 kDa for RsaFa and 50.2 kDa for RsaFb). Western immunoblot analysis demonstrated the loss of the RsaF proteins in the rsaF knockout strains (FIG. 1A). Densitometry analysis of the Western blots showed that the levels of the remaining RsaF protein in the single rsaF knockouts were the same as those observed in the wild-type strains.

Levels of RsaA secreted by the rsaF knockout strains were analyzed. When low pH extracted protein levels (representing secreted protein) of knockout and wild-type strains were compared, there was a progressive decrease in amounts of RsaA secreted as the rsaF genes were lost (FIG. 1B). Disrupting rsaFa decreased S-layer secretion to a greater extent than loss of rsaFb, but both single rsaF mutants were still capable of secreting RsaA. Elimination of both rsaF genes (JS1009), however, led to a complete loss of RsaA secretion. Levels of RsaA secretion could not be easily determined by SDS-PAGE and Coomassie staining, as small amounts of RsaA were not readily detected, therefore Western immunoblotting was performed.

Quantification of S-layer secretion levels by chemiluminescence Western blotting showed that disruption of rsaFb decreased RsaA secretion to 75.6±1.59% of wild-type levels whereas loss of rsaFa decreased RsaA secretion to 54.5±1.49% compared to wild-type NA1000 levels (Table 3). RsaA in the rsaF double knockout (4.44±1.74% of wild-type) was only detectable with Western blotting and was thought to be due to release of RsaA from cells that burst during the low pH extraction.

To confirm that apparent RsaA secretion in JS1009 was due to burst cells during low pH extraction, whole-cell preparations were analyzed to determine levels of RsaA inside the cells. To ensure that only internal RsaA was analyzed, surface protein was extracted from the cell surface (as outlined in Example 1, Materials and Methods) before whole-cell-protein preparations were performed. Colorimetric Western blots show levels of internal RsaA (FIG. 1C). Levels of RsaA observed by whole-cell preparation of the double rsaF knockout appeared to be higher than those seen where the S-layer was removed by low pH extraction. RsaA levels in JS1009 were similar to levels seen in the JS1001 strain, an S-layer shedding strain, which was filtered and washed by centrifugation to remove secreted RsaA aggregates. Both the JS1009 and the JS1001 internal RsaA levels were lower than those seen in the NA1000 parent strain. The levels of RsaA in the JS1001 strain were only internal levels of S-layer, as transported RsaA was shed and removed suggesting that the RsaA detected in the double rsaF knockout was located internally. Furthermore, immunofluorescence studies of whole JS1009 cells using an anti-RsaA antibody did not detect the presence of any RsaA on the surface.

TABLE 3
RsaA levels compared to those of the wild type Determined
by whole-culture preparations or low-pH extraction^{a}

% RsaA relative to wild type^{b}

| Strain | Low pH extracted | Whole culture |
|---|---|---|
| NA1000 | 100 | 100 |
| JS1008 | 75.6 ± 1.59 | 79.7 ± 3.36 |
| JS1007 | 54.4 ± 1.49 | 56.4 ± 0.47 |
| JS1009 | 4.44 ± 1.74 | 9.56 ± 2.16 |
| JS1003 | 0 ± 0.09 | 0 ± 0.09 |
| JS1009 rsaFa | 78.5 ± 2.18 | 80.0 ± 1.22 |
| JS1009 rsaFb | 56.2 ± 2.08 | 57.3 ± 0.83 |
| JS1001 | NA | 94.8 ± 1.08 |

^{a}Spot densitometry of chemiluminescent Western blots was done at least three times with polyclonal anti-188/784 antibodies and used to determine the relative levels of RsaA produced by the cells.
Levels determined by densitometry were compared to wild-type NA1000 RsaA levels. All low-pH and whole-culture protein Preparations were normalized prior to running samples.
^{b}Values are means ± standard deviations.
NA, not applicable.

### EXAMPLE 4

### Complementation of the Secretion Deficient JS1009 Strain

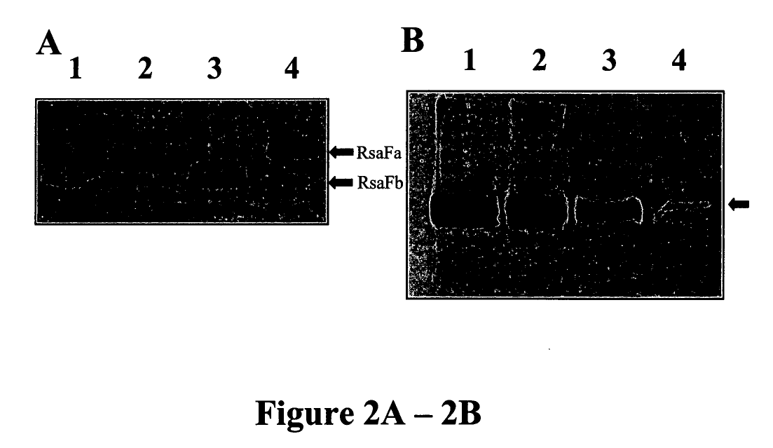

To demonstrate that the rsaF knockouts were responsible for the reduction or loss of the S-layer secretion, a multiple-copy broad-host-range plasmid containing either rsaFa (pBBR4: rsaFa) or rsaFb (pBBR4: rsaFb) was used to complement the double-knockout strain. To determine whether the plasmid borne rsaF genes were expressed, whole-culture preparations were made and were analyzed in Western Blot using polyclonal antibodies against RsaFa. Western blotting showed that the plasmid derived RsaF proteins were in fact overexpressed, at levels 9.72+0.71 (RsaFa) and 8.01±0.66 (RsaFb) fold more than wild-type strains (FIG. 2A).

Despite overexpression mediated by multicopy vectors, in both cases complementation of the individual rsaF genes only restored S-layer secretion to levels comparable to that seen with the corresponding single rsaF knockouts (FIG. 2B). The levels of RsaA secretion in the JS1009: rsaFa and JS1009: rsaFb strains were restored to levels that were only about 2% greater than those seen in the single rsaF knockouts (Table 3). Due to the lack of additional antibiotic selection markers, complementation of both rsaF genes in the double knockout was not done.

### EXAMPLE 5

### Production of RsaA was Regulated when Secretion was Impeded

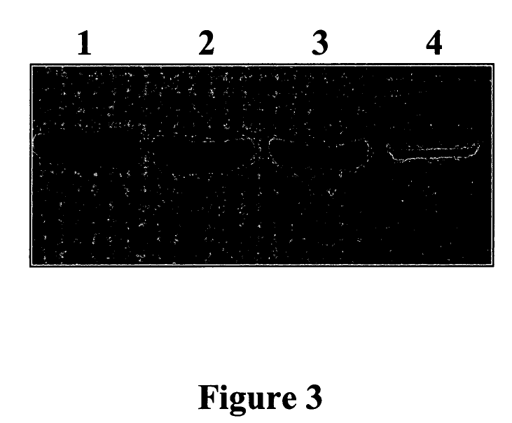

The production of RsaA in whole-culture-protein preparations was analyzed when secretion was inhibited in the RsaF mutants. Densitometry performed on Western blots showed that the levels of RsaA in the whole-culture preparations were almost identical with those observed through low pH extraction (FIG. 3) with the rsaFb knockout expression of RsaA at 79.7±3.36% of wild-type levels, and the rsaFa knockout expressing 56.4±0.47% of wild-type levels (Table 3). However, the levels of RsaA seen with the double knockout strain were higher (9.56±2.16% of wild-type NA1000 levels) in the whole-culture preparations than those seen by low pH extraction (4.44±1.74% of wild-type levels).

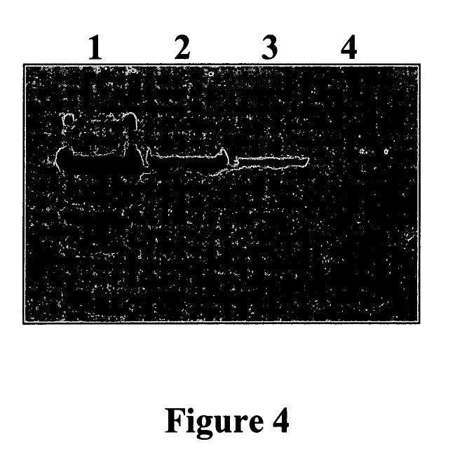

These results suggested that there was regulation of RsaA production in the cell when transport was impeded. To examine this further, the secretion of a recombinant RsaA with the charged RKKR furin cleavage sequence inserted at amino acid 723 (723/furin) was expressed in the various mutants. When this recombinant protein was expressed in JS1003, an S-layer negative strain, no recombinant protein was secreted, apparently due to the inserted positive charge cluster. Internal RsaA levels in the transporter mutants and JS1003: pWB9: 723/furin were similar to those seen in the RsaF double-knockout strain (FIG. 4): 5% of wild-type levels in the strain expressing this modified RsaA, compared to 9% for the RsaF double knockout strain. There were no apparent breakdown products seen in any of the transporter or modified RsaA mutants. Both the JS1009 rsaF strain and the JS1003: pWB9:723/furin strains grew much slower, with generation times of 114 and 108 min, respectively, compared to a generation time of 78 mm for JS1003 and the wild-type NA1000 strain, suggesting that retention of residual RsaA inside the cell was deleterious.

### EXAMPLE 6

### Over-Expression of RsaA and RsaF

Because RsaF expression could be significantly increased in trans complementation experiments, the following experiments were conducted to determine whether the number of transporter units or the amount of RsaF was a limiting factor in the amount of RsaA that was secreted. Vector borne copies of a single rsaF gene were inserted into S-layer positive C. crescentus cells to determine if levels of RsaA secretion could be increased. An S-LPS negative strain, JS1001, was used instead of the wild-type NA1000 strain to ensure that there was no possibility of RsaA regulation by surface crystallization, i.e., the possibility that RsaA secretion was impeded by full coverage of the bacterial surface. Levels of RsaA were determined by Western blot analysis of whole-culture preparations to ensure that all secreted RsaA was included in the sample.

Levels of RsaA production were not significantly increased when additional copies of rsaFa or rsaFb were expressed in JS1001 by addition of pBBR4: rsaFa or pBBR4: rsaFb. Both JS1001: rsaFa and JS1001: rsaFb produced levels of RsaA only slightly greater than JS1001 and the wild-type NA1000 strain (Table 4). Levels of RsaFa and RsaFb were determined by immunoblotting and densitometry analysis to be similar to those seen in the trans complementation strains above.

TABLE 4
RsaA levels compared to those of JS1001 determined
by whole-culture preparations^{a}

| Strain | % RsaA relative to JS1001^{b} |
|---|---|
| JS1001 | 100 |
| JS1001 rsaFa | 103.3 ± 2.95 |
| JS1001 rsaFb | 102.8 ± 2.38 |
| JS1001::pWB9rsaAΔP | 100.8 ± 1.47 |
| JS1001 rsaFa::pWB9rsaAΔP | 128.3 ± 3.69 |

^{a}Spot densitometry of chemiluminescent Western blots was done in triplicate using polyclonal anti-188/784 antibodies and used to determine the relative levels of RsaA produced by the cells.
Levels determined by densitometry were compared to JS1001 RsaA levels. All whole-culture protein preparations were normalized prior to running samples.
^{b}Values are means ± standard deviations.

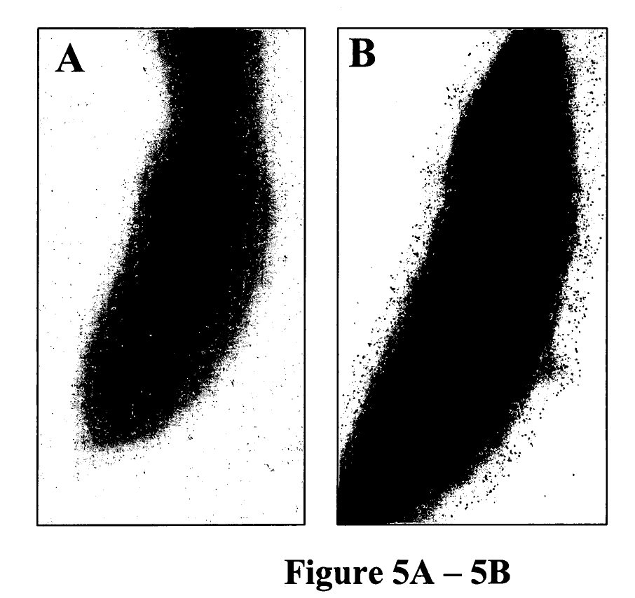

To confirm that additional RsaFa was properly targeted to the outer membrane, protein A-colloidal gold labeling with anti-RsaF antibody was used to assess levels of RsaF on the outer membrane surface. A uniform low-level labeling was observed with JS1001 (FIG. 5), indicating that some portion of the RsaF OMPs was surface exposed when the oligosaccharide chains of the S-LPS fraction of total LPS were eliminated (see below). Label of JS1001: rsaFa and JS1001: rsaFb showed two major classes of cells: those labeled at levels similar to or slightly greater than that seen with JS1001, and those (approximately 20% of the total) where a dense labeling was observed (FIG. 5). This is believed to be an indication that plasmid copy numbers for the moderate copy number plasmid pBBR4 varied significantly from cell to cell, suggesting it was not a stably maintained plasmid. Nevertheless it appeared that some cells expressed much higher levels of RsaFa and that RsaFa was targeted correctly to the outer membrane.

In contrast to JS1001 (which had no S-LPS), there was no detectable label with strain JS1003, which had no S-layer but did have a normal complement of S-LPS. Presumably this smooth form of LPS effectively blocked antibody access to the OMPs, perhaps indicating their exposure on the surface was minimal.

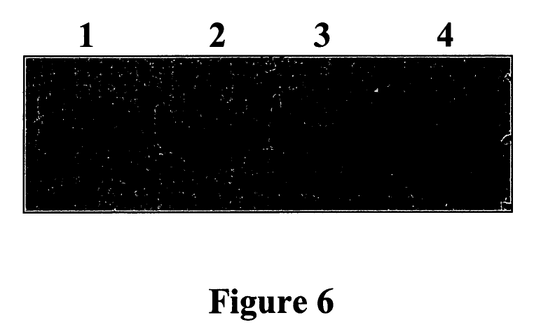

Since JS1001 had only the single chromosome resident copy of rsaA, it was considered whether, in the presence of elevated OMP levels, RsaA transcription or translation would determine the maximum levels of RsaA secretion. To address this possibility, a multi-copy plasmid-borne rsaA gene was introduced into the JS 1001:rsaFa strain, and whole-culture-protein levels were compared (FIG. 6). The resulting strain, JS1001: rsaFa: pWBrsaAΔP, produced 28.3±3.69% more S-layer protein compared to the wild-type JS1001 or JS1001: pWBrsaAΔP. Thus, elevated secretion of RsaA was achieved with overexpression of RsaA and the RsaFa OMP.

### EXAMPLE 7

### Enhanced Expression of Heterologous Proteins

A number of constructs were made to place a heterologous coding sequence in an operable linkage to a sequence coding for the secretion signal within the C-terminal region of the S-layer protein of C. crescentus. The constructs were introduced into C. crescentus strains with or without an additional vector carrying the C. crescentus rsaFa gene. The results indicate that the levels of secreted heterologous proteins were significantly enhanced by the additional expression of the rsaFa gene from the exogenous vector. See Table 5.

Plasmid Vector:

pUC8CVX:0-690—A high copy number plasmid with a control secretion signal only (the C-terminal 336 amino acids of the C. crescentus S-layer protein).

pTOPOVCM:0-690 CEX—A high copy number vector available from Invitrogen, containing an Exo-cellulase fused to a secretion signal (the C-terminal 336 amino acids of the C. crescentus S-layer protein).

pUC8CVX:0-690 PA432—A high copy number vector with domain IV of Anthrax protective antigen fused to a secretion signal (the C-terminal 336 amino acids of the C. crescentus S-layer protein).

pTOPOvcm:0-690 VP2Edelta—A high copy number vector available from Invitrogen, containing a porion of IPNV fish virus fused to a secretion signal (the C-terminal 336 amino acids of the C. crescentus S-layer protein).

pBBR4—A moderate copy number plasmid compatible with the high copy number cloning vectors. The term “compatible” means the vectors can reside in the same cell.

pBBR4:rsaFa —pBBR4 containing the C. crescentus rsaFa gene.

Bacterial Host Strain:

B5BAC=Caulobacter crescentus strain CB2B5BAC. This is an S-layer negative host (amber mutation in the S-layer gene), spontaneous holdfast (adhesion organelle at the tip of the stalk) negative and has the BAC plasmid replication genes (needed to replicate the pTOPOVCM or pUC8CVX plasmids) inserted into the recA gene.

TABLE 5

| | | | | | | | ECL Western--% rel. to | | |
|---|---|---|---|---|---|---|---|---|---|
| | | | | Aggregates | | | strains with WT RsaFa | | |
| | OD 600 | | | (total dry weight) g/L | | | expression | | |
| Strain | Trial 1 | 2 | 3 | 1 | 2 | 3 | Trial 1 | 2 | 3 |
| B5BAC (pBBR4/pUC8CVX:0-690) | 1.38 | 2.59 | 1.73 | 0.05356 | 0.1698 | 0.09128 | 100% | 100% | 100% |
| B5BAC (pBBR4:rsaFa/pUC8CVX:0-690) | 1.33 | 1.88 | 1.8 | 0.07444 | 0.2046 | 0.08272 | 124% | 141% | 101% |
| | | | | 139% | 120% | 91% | | | |
| B5BAC (pBBR4/pTOPOVCM:0-690 CEX) | 1.63 | 1.19 | 1.56 | 0.1482 | 0.1911 | 0.01326 | 100% | 100% | 100% |
| B5BAC (pBBR4:rsaFa/pTOPOVCM:0-690 C | 1.36 | 1.12 | 1.5 | 0.1983 | 0.2635 | 0.01703 | 112% | 138% | 118% |
| | | | | 134% | 138% | 128% | | | |
| B5BAC (pBBR4/pUC8CVX:0-690 PA432) | 2.6 | 1.21 | 1.27 | 0.1211 | 0.2182 | 0.08656 | 100% | 100% | 100% |
| B5BAC (pBBR4:rsaFa/pUC8CVX:0-690 PA4 | 2.3 | 1.18 | 1.05 | 0.1659 | 0.3067 | 0.1334 | 118% | 159% | 97% |
| | | | | 137% | 141% | 154% | | | |
| B5BAC (pBBR4/pTOPOvcm:0-690 VP2Edelta) | | | | | | | 100% | 100% | 100% |
| B5BAC (pBBR4:rsaFa/pTOPOvcm:0-690 | | | | | | | 85% | 158% | 170% |
| VP2Edelta) | | | | | | | | | |

OD600 = optical density of the culture (absorbance at 600 nm). Applies only to the aggregates experiments.
Aggregates - S-layer fusion proteins aggregate in the culture medium after secretion and can be collected, dried and weighed. The values were normalized to culture volume, not cell numbers.
ECL Western - A quantitative Western immunoblot method that measured all the recombinant S-layer produced by the cells. Since >90% of the protein was secreted, ECL Western was a simple way to test secretion levels. The values were normalized to cell numbers.

### EXAMPLE 8

### Integration of rsaFa into C. crescentus Genome

Oligonucleotides are designed that will hybridize to the 5′ and 3′ region of the C. crescentus xlyX gene, and contain nucleotides corresponding to selected restriction sites for convenient cloning. Polymerase Chain Reaction (PCR) amplification is performed using the above oligonucleotides as primers, and Caulobacter DNA as template, to create a cloned copy of the xlyX gene. The xylX PCR product is then cloned into an initial simple cloning vector plasmid. An internal portion of the xlyX gene is then deleted from the vector, replaced therewith a copy of the rsaFa gene, linked to an E. coli lactose operon promoter (plac).

The 5′-xlyX-plac-rsaFa-xlyX-3′ DNA segment is then cloned into the plasmid pKmobsacB. This plasmid cannot be replicated by Caulobacter strains, specifies kanamycin resistance and also contains the Bacillus sacB gene. Expression of the sacB gene is inducible by addition of sucrose to the culture medium and results in the production of a levan carbohydrate polymer. Production of the levan polymer within a bacterium is lethal.

The plasmid, pKmobsacB: 5′-xlyX-plac-rsaFa-xlyX-3′, is introduced into Caulobacter by electroporation. Because the plasmid cannot be replicated, only those copies of the plasmid that is integrated into the chromosome by homologous recombination persist when kanamycin drug selection is applied. Because recombination is nearly always homologous, the plasmid will have crossed into the chromosome at a region corresponding to the 5′ or the 3′ end of the xylX gene (i.e., these regions are contained on the plasmid vector). A kanamycin resistant clone is selected and grown followed by exposure to 3-5% sucrose addition to the medium. Only those cells that have excised the plasmid by recombination (and thereby stop producing levan) will survive. The excision event can occur anywhere along the regions of homology between the original incoming event and the chromosomal xylX gene. Two phenotypes are possible: a) simple excision of the incoming plasmid and restoration of the original chromosomal condition (this is unwanted); or b) replacement with the modified gene containing the inserted plac-rsaFa segment. In either case, sensitivity to kanamycin will be restored. Determining which event has occurred can be accomplished in several ways. One test is to assess loss of the ability to grow on xylose as sole carbon source. Additional confirmation achieved by measuring an increased production of RsaFa protein by western immunoblot methods, using antisera raised against the RsaFa protein.

### REFERENCES

1. Altschul, S. F., T. L. Madden, A. A. Schaffer, J. Zhang, Z. Zhang, W. Miller, and D. I. Lipman 1997. Gapped BLAST and PSI-BLAST: a new generation of protein database search programs. Nucleic Acids Res. 25:33 89-3402.
2. Andersen, C. 2003. Channel-tunnels: outer membrane components of type I secretion systems and multidrug efflux pumps of Gram-negative bacteria. Rev. Physiol. Biochem. Pharmacol. 147:122-165.
3. Awram, P, and J. Smit 1998. The Caulohacter crescentus paracrystalline S-layer protein is secreted by an ABC transporter (Type 1) secretion apparatus. J. Bacteriol. 180:3062-3069.
4. Awram, P., and J. Smit 2001. Identification of lipopolysaccharide O-antigen synthesis genes required for attachment of the S-layer of C. crescentus. Microbiology. 147:1451-1460.
5. Beveridge, T. J., P. H. Pouwels, M. Sara, A. Kotiranta, K. Lounatmaa, K. Karl, E. Kerosuo, M. Haapasalo, E. M. Egelseer, I. Schocher, U. B. Sleytr, L. Morelli, M. L. Callegari, J. F. Nomellini, W. H. Bingle, J. Smit, E. Leibovitz, M. Lemaire, I. Miras, S. Salamitou, P. Beguin, H. Ohayon, P. Gounon, M. Matuschek, K. Sahm, H. Bahl, R. Grogono-Thomas, J. Dworkin, M. J. Blaser, R. M. Woodland, D. G. Newell, M. Kessel, and S. F. Koval 1997. Functions of S-layers. FEMS Microbiol. Rev. 20:99-149.
6. Binet, R., S. Letoffe, J. M. Ghigo, P. Delepelaire, and C. Wandersman 1997. Protein secretion by gram-negative bacterial ABC transporters-a review. Gene 192:7-11.
7. Binet, R., and C. Wandersman 1996. Cloning of the Serratia marcescens hasF gene encoding the Has ABC exporter outer membrane component: a TolC analogue. Molec. Microbiol. 22:265-273.
8. Bingle, W. H., H. D. J. Kurtz, and J. Smit 1993. An “all-purpose” cellulase reporter for gene fusion studies and application to the paracrystalline surface (S)-layer protein of Caulobacter crescentus. Can. J. Microbiol. 39:70-80.
9. Bingle, W. H., J. F. Nomellini, and J. Smit 1997. Cell-surface display of a Pseudomans aeruginosa strain K pilin peptide within the paracrystalline S-layer of Caulobacter crescentus. Molec. Microbiol. 26:277-288.
10. Bingle, W. H., J. F. Nomellini, and J. Smit 1997. Linker mutagenesis of the Caulobacter crescentus S-layer protein: toward a definition of an N-terminal anchoring region and a C-terminal secretion signal and the potential for heterologous protein secretion. J. Bacteriol. 179:601-611.
11. Bingle, W. H, J. F. Nomellini, and J. Smit 2000. Secretion of the Caulobacter crescentus S-layer protein: further localization of the C-terminal secretion signal and its use for secretion of recombinant proteins. J. Bacteriol. 182:3298-3301.
12. Blight, M. A., and I. B. Holland 1994. Heterologous protein secretion and the versatile Escherichia coli haemolysin translocator. Trends Biochem. Sci. 12: 450-455.
13. Duong, F., E. Bonnet, V. Geli, A. Lazdunski, M. Murgier, and A. Filloux 2001. The AprX protein of Pseudornonas aeruginosa: a new substrate for the Apr type I secretion system. Gene. 262:147-153.
14. Edwards P., and J. Smit 1991. A transducing bacteriophage for Caulobacter crescentus uses the paracrystalline surface layer protein as a receptor. J. Bacteriol. 173:5568-5572.
15. Fellay, R., J. Frey, and H. Krisch 1987. Interposon mutagenesis of soil and water bacteria: a family of DNA fragments designed for in vitro insertional mutagenesis of gram-negative bacteria. Gene 2: 147-154.
16. Fernandez-Hlerrero, L. A., G. Olabarria, and J. Berenguer 1997. Surface proteins and a novel transcription factor regulate the expression of the S-layer gene in Therrnus Thermophiius HB8. Molec. Microbiol. 24:61-72.
17. Fisher, J., J. Smit, and N. Agabian 1988. Transcriptional analysis of the major surface aray gene of Caulobacter crescentus. J. Bacteriol. 170:4706-4713.
18. Fralick, J. A. 1996. Evidence that TolC is required for functioning of the Mar/AcrAB efflux pump of Escherichia coli. J. Bacteriol. 178:5803-5805.
19. Gentschev, I,, G. Dietrich, and W. Goebel 2002. The E coli alpha-haemolysin secretion system and its use in vaccine development. Trends in Microbiol. 10:39-45.
20. Gilchrist, A., and J. Smit 1991. Transformation of freshwater and marine Caulohacters by electroporation. J. Bacteriol. 73:921-925.
21. Hahn, H. P., and B. U. von Specht 2003. Secretory delivery of recombinant proteins is attenuated Salmonella strains: potential and limitations of Type I protein transporters. FEMS Immunol. Med. Micro. 37:87-98.
22. Hwang, J. W., X. T. Zhong, and P. C. Tai 1997. Interactions of dedicated export membrane proteins of the colicin V secretion system: CvaA, a member of the membrane fusion protein family, interacts with CvaB and TolC. J. Bacteriol. 179:6264-6270.
23. Kahala, M., K. Savijoki, and A. Palva 1997. In vivo expression of the Lactobacillus S-layer gene. J. Bacteriol. 179:284-286.
24. Kawai, E., H. Akatsuka, A. Idei, T. Shibatani, and K. Omori 1998. Serratia marcescens S-layer protein is secreted extracellularly via an ATP-binding cassette exporter, the Lip system. Molec. Microbiol. 27:941-952.
25. Koronakis, V., A. Sharff, E. Koronakis, B. Luisi, and C. Hughes 2000. Crystal structure of the bacterial membrane protein TolC central to multidrug efflux and protein export. Nature. 405:914-9.
26. Koval, S. F., and S. H. Hynes 1991. Effect of paracrystalline protein surface layers on predation by Bdeliovibrio bacteriovorus. J. Bacteriol. 173:2244-2249.
27. Letoffe, S., P. Delepelaire, and C. Wandersman 1990. Protease secretion by Erwinia chrysanthemi: The specific secretion functions are analogous to those of Escherichia coli alpha-haemolysin. EMBO J. 9:1375-1382.
28 Letoffe, S., J. M. Ghigo, and C. Wandersman 1994. Iron acquisition from heme and hemoglobin by a Serratia marcescens extracellular protein. Proc. Nat. Acad. Sci. U.S.A. 91:9876-9880.
29. Letoffe, S., and C. Wandersman 1992. Secretion of CyA-PrtB and Hy1A-PrtB fusion proteins in Escherichia coli: involvement of the glycine-rich repeat domain of Erwinia chrysanthemi protease B. J. Bacteriol. 174:4920-4927.
30. Mackman, N., J. M. Nicaud, L. Gray, and I. B. Holland 1985. Identification of polypeptides required for export of haemolysin 2001 from E. coli. Mol. Gen. Genet. 201:529-536.
31. Mollenkopf, H. J., I. Gentschev, and W. Goebel 1996. Conversion of bacterial gene products to secretion-competent fusion proteins. Biotechniques. 21:856-860.
32. Morales, V. M., A. Backman, and M. Bagdasarian 1991. A series of wide-host-range low-copy-number vectors that allow direct screening for recombinants. Gene. 97:39-47.
33. Nierman, W. C., T. V. Feldblyum, M. I. Laub, I. I. Paulsen, K. E. Nelson, .J. Eisen, J. F. Heidelberg, M. P. K. Alley, N. Olita, J. P. Maddock, I. Potocka, W. C. Nelson, A. Newton, C. Stephens, N. D. Phadke, B. Ely, R. T. Debroy, R. J. Dodson, A. S. Durkin, M. L. Gwinn, D. H. Haft, J. F. Kolonay, J. Smit, M. B. Craven, H. Khouri, J. Shetty, K. Berry, T. Utterback, K. Iran, A. Wolf, J. Vamathevan, M. Ermolaeva, O. White. S. L. Salzberg, J. C. Venter, L. Shapiro, and C. M. Fraser 2001. Complete genome sequence of Caulohacter crescentus strain CB15. Proc. Nat. Acad. Sci. U.S.A. 98:4136-4141.
34. Nomellini, J. F., M. C. Toporowski, and J. Smit 2004. Secretion or presentation of recombinant proteins and peptides mediated by the S-layer of Caulobacter crescentus, Expression Technologies: Current Status and Future Trends. Horizon Scientific Press.
35. Reichelt, M., B. U. von Specht, and H. P. Hahn 2001. The Caulobacter crescentus outer membrane protein Omp58 (RsaF) is not required for paracrystalline S-layer secretion. FEMS Microbiol. Left. 201:277-283.
36. Sambrook, 3., E. F. Fritsch, and T. Maniatis 1989. Molecular cloning: a laboratory manual Cold Spring Harbor Laboratory Press. 2nd ed.
37. Schafer A, Tauch A, Jager W, Kalinowski J, Thierbach G, Puhler A. 1994. Small mobilizable multi-purpose cloning vectors derived from the Escherichia coli plasmids pK18 and pK19: selection of defined deletions in the chromosome of Corynebacterium glutamicum. Gene 145(1):69-73.
38. Smit, J. 1987. Localizing the subunit pool for the temporally regulated polar pili of Caulobacter crescentus. J. Cell Biol. 105:1821-1828.
39. Smit, J., and N. Agabian 1982. Cell surface patterning and morphogenesis of a periodic surface array during Caulobacter development. J. Cell Biol. 95:41-49.
40. Smit, J., and N. Agabian 1984. Cloning of the major protein of the Caulobacter crecentus periodic surface layer: detection and characterization of the cloned peptide by protein expression assays. J. Bacteriol. 160:1137-1145.
41. Smit, J., H. Engelhardt, S. Volker, S. H. Smith, and W. Baumeister 1992. The S-layer of Caulobacter crescentus: three-dimensional image reconstruction and structure analysis by electron microscopy. J. Bacteriol. 174:6527-38.
42. Smit, J., D. A. Grano, R. M. Glaeser, and N. Agabian 1981. Periodic surface array in Caulobacter crescentus: fine structure and chemical analysis. J. Bacteriol. 146:1135-1150.
43. Spreng, S., G. Dietrich, S. Niewiesk, V. Meulen, I. Gentschev, and W. Goebel 2000. Novel bacterial systems for the delivery of recombinant protein or DNA. FEMS Immunol. Med. Micro. 27:299-304.
44. Thanabalu, I., E, Koronakis, C. Hughes, and V. Koronakis 1998. Substrate-induced assembly of a contiguous channel for protein export from E. coli: reversible bridging of an inner-membrane translocase to an outer membrane exit pore. EMIBO J. 17:6487-6496.
45. Thanassi, D. G., and S. J. Hultgren 2000. Multiple pathways allow protein secretion across the bacterial outer membrane. Curr. Opin. Cell Biol. 12:420-430.
46. Thompson, S. A., 0. I. Shedd, K. C. Ray, M. H. Reins, J. P. Jorgensen, and M. J. Blaser 1998. Campylobacter fetus surface layer proteins are transported by a type I secretion system. J. Bacteriol. 180:6450-6458.
47. Umelo-Njaka, E., W. H. Bingle, F. Borchani, K. H. Le, P. Awram, T. Blake, J. F. Nomellini, and J. Smit 2002. Caulobacter crescentus synthesizes an S-layer editing metalloprotease possessing a domain sharing sequence similarity with its paracrystailine S-Layer protein. J. Bacteriol. 184:2709-2718.
48. Walker, S., H. N. Kurunaratne, N. Ravenscroft, and J. Smit 1994. Characterization of mutants of Caulobacter crescentus defective in surface attachment of the paracrystalline surface layer. J. Bacteriol. 176:6312-6323.
49. Walker, S., S. H. Smith, and J. Smit 1992. Isolation and comparison of the paracrystalline surface layer proteins of freshwater Caulobacters. J. Bacteriol. 174: 1783-1792.
50. Wandersman, C., and P. Delepelaire 1990. TolC, an Escherichia coli outer membrane protein required for hemolysin secretion. Proc. Nat. Acad. Sci. U.S.A. 87:4776-4780.

What is claimed is:

1. A vector for expressing a RsaFa or RsaFb protein in Caulobacter, comprising a nucleotide sequence coding for said RsaFa or RsaFb protein.

2. The vector of claim 1, wherein said nucleotide sequence is operably linked to a promoter functional in said Caulobacter.

3. The vector of claim 1, wherein said vector is a replicative plasmid.

4. The vector of claim 1, wherein said vector is an integrative vector.

5. The vector of claim 1, wherein said RsaFa or RsaFb protein is from C. crescentus.

6. The vector of claim 1, wherein said nucleotide sequence codes for a protein as set forth in SEQ ID NO: 2 or SEQ ID NO: 4.

7. The vector of claim 1, wherein said nucleotide sequence comprises the sequence as set forth in SEQ ID NO: 1 or SEQ ID NO: 3.

8. A genetically engineered Caulobacter strain, which produces at least one of RsaFa or RsaFb at an elevated level.

9. The strain of claim 8, which produces RsaFa at an elevated level.

10. The strain of claim 8, which produces RsaFa and RsaFb at elevated level.

11. The strain of claim 8, comprising a vector that comprises a nucleotide sequence coding for said RsaFa or said RsaFb, operably linked to a promoter.

12. The strain of claim 8, comprising a nucleotide sequence coding for said RsaFa or said RsaFb, which is operably linked to a promoter, wherein said nucleotide sequence coding for said RsaFa or said RsaFb and said promoter are integrated in the chromosome of said strain.

13. The strain of claim 8, wherein said strain is a strain of C. crescentus.

14. The strain of claim 13, wherein said RsaFa comprises SEQ ID NO: 2 and said RsaFb comprises SEQ ID NO: 4.

15. The strain of claim 8, further comprising a nucleotide sequence coding for a fusion protein, wherein said fusion protein is a fusion of a heterologous polypeptide and at least a portion of a Caulobacter S-layer protein which comprises a secretion signal of said S-layer protein.

16. The strain of claim 15, wherein said S-layer protein is an S-layer protein of C. crescentus.

17. The strain of claim 15, wherein said secretion signal comprises the C-terminal 82 amino acids of said S-layer protein.

18. A method of producing a heterologous polypeptide in Caulobacter, comprising transforming a genetically engineered Caulobacter strain which produces at least one of RsaFa or RsaFb at an elevated level, with a nucleotide sequence coding for a fusion protein, wherein said fusion protein is a fusion of said heterologous polypeptide and at least a portion of a Caulobacter S-layer protein which comprises a secretion signal of said S-layer protein; and producing said heterologous polypeptide from said strain.

19. A method of producing a heterologous polypeptide in Caulobacter, comprising obtaining a Caulobacter strain which comprises a nucleotide sequence coding for a fusion protein, wherein said fusion protein is a fusion of said heterologous polypeptide and at least a portion of a Caulobacter S-layer protein which comprises a secretion signal of said S-layer protein; transforming said strain with a nucleotide sequence coding for at least one of RsaFa or RsaFb, operably linked to a promoter; and producing said heterologous polypeptide from said strain.

20. The method of claim 19, wherein said nucleotide sequence coding for at least one of RsaFa or RsaFb is on a plasmid that remains episomal in said strain.

21. The method of claim 19, wherein said nucleotide sequence coding for at least one of RsaFa or RsaFb is on an integrative vector that integrates into the chromosome of said strain after transformation.


SEQUENCE LISTING (21 sequences)

SEQ ID NO: 1 - DNA, 1584 bp, Caulobacter crescentus
atgcgagtgctgtcgaaagttctgtccgtgcgaacgtctctgatcgccttggccatggccatggcggtcgtcggtcgcgctgatctcgcccacgccgagaccttggccgaggcgatcaccgcagcctatcagagcaatccgaatattcaggcccaacgcgccgccatgcgcgcgctggacgagaactacacccaggcccgttcggcctatgggctgcaagccagcgcctcggtcgctgaggtctatggctggtccaagggcgtcaacgccaagaacggcgtcgaggccgccagccagacctcgaccctctctctgagccagagcctctacaccaacggtcgtttctcggcccgcctggcgggtgtcgaggcgcagatcaaggccgcgcgcgagaacctgcgccgcatcgagatggacctgctggtccgcgtgaccaacgcctatatctcggtgcgccgcgaccgcgagatcctgcggatcagccaaggcggtgaagcctggctgcagaagcaattgaaggacaccgaggacaagtacagcgtccgtcaggtgaccttgaccgacgtgcagcaggccaaggcccgcctggcgtcggccagcactcaggtggcgaacgcccaggcgcagctgaatgtcagcgtagcgttctacgcgtccctggtggggcgccagccggagacgctggagcctgaacccgatattgacggcctgcctacaaccctcgacgaggcgttcaatcaggccgaacaagccaatccggtcctgctggcggcgggctacaccgagaaggcctctcgcgccggcgtcgccgaggcgcgggcccagcgcctgttctcggtcggcgcgcgcgcggactatcgcaatggctccagcacgccgtactacgcgcgtggcggtctgcgcgaggacaccgtcaacgcctcgatcaccctgacccagccgctgttcaccagcggtcagctgaacgcctcggtgcggcagtcgatcgaggagaacaaccgcgacaagctgctgatggaagacgcacgtcgcagcatggtcctgagcgtctcgcagtactgggacagcctggtggccgcgcggaagtcgctggtcagcctcgaagaggaaatgaaggccaacacgatcgccttctatggggtgcgcgaagaagagcgtttcgcgcttcgcagcacgatcgaagtgctgaacgcccaagccgaattgcagaacgcccagatcaatttcgtccgcgggcgcgccaacgagtatgtcggtcggctccatcttctggcgcaggtcggcacgcttgaggtcggcaatctcgctcccggcgtccagccctacgatcctgagcgtaacttcaggaaggtccggtaccgcggcgctttgccgacggagctgatcatcggaaccttcgacaagatcgccttgccgctcgagcccaagaagccggcgccgggggacacctcgccgatccggccgccgtcgagcgaactgccggccaggcctgtttcggccgacaaggtgacgccgccggcgtcgatgaacgatctgcccgccctgaccgacgacacgccggtccagaccgcgccccgcaactag

SEQ ID NO: 2 - protein, 527 aa, Caulobacter crescentus
MRVLSKVLSVRTSLIALAMAMAVVGRADLAHAETLAEAITAAYQSNPNIQAQRAAMRALDENYTQARSAYGLQASASVAEVYGWSKGVNAKNGVEAASQTSTLSLSQSLYTNGRFSARLAGVEAQIKAARENLRRIEMDLLVRVTNAYISVRRDREILRISQGGEAWLQKQLKDTEDKYSVRQVTLTDVQQAKARLASASTQVANAQAQLNVSVAFYASLVGRQPETLEPEPDIDGLPTTLDEAFNQAEQANPVLLAAGYTEKASRAGVAEARAQRLFSVGARADYRNGSSTPYYARGGLREDTVNASITLTQPLFTSGQLNASVRQSIEENNRDKLLMEDARRSMVLSVSQYWDSLVAARKSLVSLEEEMKANTIAFYGVREEERFALRSTIEVLNAQAELQNAQINFVRGRANEYVGRLHLLAQVGTLEVGNLAPGVQPYDPERNFRKVRYRGALPTELIIGTFDKIALPLEPKKPAPGDTSPIRPPSSELPARPVSADKVTPPASMNDLPALTDDTPVQTAPRN

SEQ ID NO: 3 - DNA, 1452 bp, Caulobacter crescentus
atgttgatgtcgaaccgtcgacgggccgggttgctggccgccgcttgcagcgctggtcttctggccgggtccttctccatcgcgaacgccgagactctcgccgatgcgatcgcgctcgcctatcagaccaatccgacgctgcagcaacagcgcgccagcgcccgcatcaccgacgaaggcgtggtgcaagccaagaccggcttccgcccgaccgtgggcgccaccgccgatatcacgggggcgaacaccaatccggccagcgccggcgcggatgtgaagctctctggcagcagcgcggcgctttcgatcatccagccgctctacaccggcggtcgcgccagcgcgggcgtcagcgccgctgaagccgacgtgctgtctgcgcgggaaggtcttcgcgcggtcgagcagggggtgctggtcagcgtcgtccaggcctatgtcgacgtgcgccgagaccaggaacgcctgcgcatcgccaaggaaaacgtcgcggttctgcagcgccagctcgaagaatcgaacgctcgcttcgacgtgggtgagatcacccggacggacgtcgcccagtctcaggcgcgcttggcttcggccaaggccagcctgtcgggcgcccaggcccagttggaagtcagccgcgcctcctacgctgcggtggtcggtcaaacgcccggcgaactggctcccgagccgagcttggccggactgctgcccgccagcgtcgaccaggctttcgaggccgcccagacgaacaatcctggcgtgacctcggcgaagttcaatgaagaggcggccgccgcgcgtgtcgccgtcgccaaggcgggctatcgaccgacggtctcggcgcgcgcttcgctgggttacgatgcgacgcgtcgttctggcgtcggcagccagtttgacgactattcgcgcaccgtttcgggcgcgatcacggcctcgatcccgcttttcgccggcggcttgaccacgtcgcaggtccgggcggccaatgaacgtgagagcgctgcgcgcagcgccgtcgaaggcgccaagcgcaccgcgatccagcaagtctcgaacgcgtggagcaatctgctggcgtcgcgcgccggtctggtcgccaacgaggagcaggttcgagcgacccgcatcgctttcgaaggcgtgcgtcaggagcaacaggtcggcctgcggacgacgctggacgttctgaacgcccagctggagctgtccaacgccgaactggcgctggtttcggcccgtcgcgacgaatacctggcttccgccgtggtcttgcaggccatgggccaactggaggtgaccaagctcgcctccaacaccgccgcctatgacccgaaaacctcctacaaccgcgtgacgcgcggcgtgggctgggtgccgtgggagccggtggtgcaggcgatcgacagcgtcggcgcggcttccaccaagcccccgagcggctcgaaatag

SEQ ID NO: 4 - protein, 483 aa, Caulobacter crescentus
MLMSNRRRAGLLAAACSAGLLAGSFSIANAETLADAIALAYQTNPTLQQQRASARITDEGVVQAKTGFRPTVGATADITGANTNPASAGADVKLSGSSAALSIIQPLYTGGRASAGVSAAEADVLSAREGLRAVEQGVLVSVVQAYVDVRRDQERLRIAKENVAVLQRQLEESNARFDVGEITRTDVAQSQARLASAKASLSGAQAQLEVSRASYAAVVGQTPGELAPEPSLAGLLPASVDQAFEAAQTNNPGVTSAKFNEEAAAARVAVAKAGYRPTVSARASLGYDATRRSGVGSQFDDYSRTVSGAITASIPLFAGGLTTSQVRAANERESAARSAVEGAKRTAIQQVSNAWSNLLASRAGLVANEEQVRATRIAFEGVRQEQQVGLRTTLDVLNAQLELSNAELALVSARRDEYLASAVVLQAMGQLEVTKLASNTAAYDPKTSYNRVTRGVGWVPWEPVVQAIDSVGAASTKPPSGSK

SEQ ID NO: 5 - DNA, 29 bp, Artificial Sequence
  Oligonucleotide Primer EEH-1
cgcgctgaattcggatatcttaagcttgg

SEQ ID NO: 6 - DNA, 29 bp, Artificial Sequence
  Oligonucleotide primer EEH-2
cgcgccaagcttaagatatccgaattcag

SEQ ID NO: 7 - DNA, 29 bp, Artificial Sequence
  Oligonucleotide primer ESH-1
cgcgctgaattcgaggcctttaagcttgg

SEQ ID NO: 8 - DNA, 29 bp, Artificial Sequence
  Oligonucleotide primer ESH-2
cgcgccaagcttaaagggctcgaattcag

SEQ ID NO: 9 - DNA, 35 bp, Artificial Sequence
  Oligonucleotide primer rsaFa-1
cgcggatccatgcgagtgctgtcgaaagttctgtc

SEQ ID NO: 10 - DNA, 33 bp, Artificial Sequence
  Oligonucleotide primer rsaFa-2
ccgggaattctagttgcggggcgcggtctggac

SEQ ID NO: 11 - DNA, 34 bp, Artificial Sequence
  Oligonucleotide primer rsaFb-1
cgcggatccatgttgatgtcgaaccgtcgacggg

SEQ ID NO: 12 - DNA, 33 bp, Artificial Sequence
  Oligonucleotide primer rsaFb-2
ccgggaattctatttcgagccgctcgggggctt

SEQ ID NO: 13 - DNA, 18 bp, Artificial Sequence
  Oligonucleotide primer rsaFbNC-1
gaagccgacgtgctgtct

SEQ ID NO: 14 - DNA, 20 bp, Artificial Sequence
  Oligonucleotide primer rsaFbNC-2
tgtaggaggttttcgggtca

SEQ ID NO: 15 - DNA, 36 bp, Artificial Sequence
  Oligonucleotide primer 1060
gaggcctagtactctgtcagaccaagtttactcata

SEQ ID NO: 16 - DNA, 33 bp, Artificial Sequence
  Oligonucleotide primer 1920
gaggcctactcttcctttttcaatattattgaa

SEQ ID NO: 17 - DNA, 36 bp, Artificial Sequence
  Oligonucleotide primer CHE-1
ggaagatctgttaacttttcaggagctaaggaagct

SEQ ID NO: 18 - DNA, 38 bp, Artificial Sequence
  Oligonucleotide primer CHE-2
gaagatctgttaacacaataactgccttaaaaaaatta

SEQ ID NO: 19 - DNA, 26 bp, Artificial Sequence
  Oligonucleotide primer RKKR-1
tcgagacccgatgcgcaagaaacggg

SEQ ID NO: 20 - DNA, 22 bp, Artificial Sequence
  Oligonucleotide primer RKKR-2
cccgtttcttgcgcatcgggtc

SEQ ID NO: 21 - DNA, 27 bp, Artificial Sequence
  Oligonucleotide primer rsaFb-I90
ggacgacgctgaccagcaccccctgct